(19) United States
(12) Patent Application Publication
(10) Pub. No.: US 2025/0058027 A1
(43) Pub. Date: Feb. 20, 2025

(54) Nanoyarns as Drug-Releasing Facilitator

(71) Applicant: Amrita Vishwa Vidyapeetham, Kerala (IN)

(72) Inventors: Deepthy Menon, Kerala (IN); Binulal Nelson Sathy, Kerala (IN); Priya Nair, Kerala (IN); Rosebin Babu, Kerala (IN); Kavitha Gopal, Kerala (IN); Shantikumar V. Nair, Kerala (IN)

(73) Assignees: Amrita Vishwa Vidyapeetham, Kerala (IN); Texnano Technologies PVT LTD, Kerala (IN)

(21) Appl. No.: 18/527,772

(22) Filed: Dec. 4, 2023

(30) Foreign Application Priority Data
Aug. 17, 2023 (IN) 202341055305

(51) Int. Cl. A61L 31/02; A61L 31/16

(52) U.S. Cl. CPC A61L 31/022; A61L 31/16; A61L 2300/216; A61L 2300/232; A61L 2300/416; A61L 2300/602; A61L 2400/12

(57) ABSTRACT

A self-expanding metallic stent (SEMS) has drug-encapsulated polymeric yarns intertwined with stent struts at specific locations along a length of the stent. The polymeric yarns in one instance comprise microfibers.

### CROSS REFERENCE TO RELATED APPLICATIONS

The present application claims priority to India Patent Application 202341055305 filed 17 Aug. 2023. All disclosure of the parent case is incorporated herein at least by reference.

### BACKGROUND OF THE INVENTION

### 1. Field of the Invention

The present invention is in the technical field of cancer treatment apparatus and pertains more particularly to nanoyarn for delivering cancer treatment medication.

### 2. Description of Related Art

Self-expanding metallic stents (SEMS) and plastic stents are routinely used in clinics for biliary stenting as the primary standard of care for unresectable malignant biliary obstruction, by way of palliative treatment. Of these, SEMS are preferred because they do not need to be replaced every 3-4 months and repeat procedures can be avoided.

However, even SEMS lack long-term patency due to biliary sludge deposition, and tumor ingrowth or overgrowth. Biliary sludge deposition is mainly cleared by inserting plastic stents with or without ablation. This involves multiple re-interventions that offer risk elements to the patient's health and increases the cost of the procedure. Biliary draining in patients with malignant biliary obstruction is vital, because obstructive jaundice precludes the administration of palliative chemotherapy (e.g.: Gemcitabine, Paclitaxel, 5-Fluorouracil, Cisplatin, Oxaliplatin, etc.), which is the only therapeutic option to reduce tumor growth. Hence there is a critical clinical need for a technique that can address stent block by bile sludge and provide sustained drug release within the bile duct for mitigating tumor ingrowth.

SEMS block is relatively common, with rates between 5.7-42.1% [1]. The two predominant causes of stent block are tumor ingrowth/overgrowth and sludge deposition. Tumor ingrowth is more common in uncovered SEMS than fully covered SEMS, because the open mesh in uncovered SEMS allows for easy tissue penetration into the SEMS [2].However, stent migration is more common with covered SEMS. Also, issues with block of bile flow from the cystic duct and biliary radicles leads to cholecystitis and cholangitis [3]. Hence, covered SEMS are not a routine choice in clinics.

Sludge deposition is a major drawback with uncovered SEMS in bile ducts. Bile glycoprotein mucin plays an important role in pigment and cholesterol-gallstone formation, acting as a pro-nucleating agent for cholesterol monohydrate crystals in bile and as a scaffold for the deposition of crystals during the growth of stones [4]. Studies have shown that the same mucin is also responsible for forming a biofilm over the inner surface of the SEMS through protein adsorption, which then acts as a nidus for bacterial colonization, sludge formation and subsequent cholangitis [5].

The present invention involves the development of an uncovered SEMS coupled with Gemcitabine-eluting nanofibrous yarn affixed to the stent struts along its length, so as to retain the nanoyarn during use, allow for stent expansion and maintain stent functionality. Gemcitabine-eluting nanoyarn provides a sustained low-dose drug release at the target site, while maintaining the porous nature of the SEMS. This nanoyarn integrated SEMS does not cause any obstruction to the bile flow from the cystic and pancreatic ducts, nor does the localized elution of Gemcitabine cause any toxicity. This strategy will address the specific issues related to early and late biliary stent occlusion to establish stent patency for a considerably longer period, in patients with malignant biliary obstruction.

The current gold standard for malignant biliary obstruction in clinics is the use of uncovered bare metal SEMS that improve the patency of obstructed bile ducts. However, biliary sludge deposition and eventual tumor in growth within these SEMS compromises its long-term patency.

Varieties of stent types that focus mainly on design/shape modification and drug elution have been investigated to partially overcome current drawbacks of biliary stents. Recent work in this area has demonstrated improved benefits for drug-incorporated polymer-coated SEMS over normal SEMS regarding its patency and rate of restenosis [6,7]. U.S. Pat. No. 9,301,926 B2 [8] describes the invention of a drug-eluting device having a film of thickness from 2 to 1000 μm made of a biodegradable polymer encapsulating chemotherapeutic drugs by solvent evaporation, showing a drug release profile up to 90 days. U.S. Pat. No. 8,999,945 B2 [9] also describes the fabrication of a drug-eluting matrix having drug-loaded polymeric fibers via electrospinning, showing a drug release profile of 3 months.

However, all these prior arts have established the effectiveness of localized drug delivery, though the drug release profiles were only for a shorter duration of up to 3 months. The use of such drug eluting stents in the common bile duct can affect stent patency owing to occlusion at later time periods. Hence to address such limitations, it is important to provide localized drug delivery to the target site and prolong the drug release for sustained durations.

Current research addresses most of these limitations through use of biodegradable polymeric nanoyarns. While the method of making a nanoyarn has been patented by us U.S. Pat. No. 9,994,975 [10], this patent seeks to use the yarn for the express purpose of this application. The novelty of its use in the application is as follows. (i) Each nanoyarn consists of thousands of nanofibers within which the drugs are encapsulated. The very high surface area and the geometrical constraint of the yarn extends drug release times considerably to up to 6 months. (ii) The nanoyarns are considerably stronger than the individual nanofibers and can easily accommodate the stent crimping and deployment, without breakage. (iii) This uncovered SEMS integrated with nanoyarns along its length provide long-term patency without biliary sludge deposition.

### BRIEF SUMMARY OF THE INVENTION

In one embodiment of the invention a self-expanding metallic stent (SEMS) is provided with drug-encapsulated polymeric yarns intertwined with stent struts at specific locations along a length of the SEMS. In one embodiment the polymeric yarns comprise biodegradable polymers including one or a combination of polycaprolactone, polydioxanone, and polyurethane. Also, in one embodiment the polymeric yarns have nanofibers loaded with chemotherapeutic drugs, including one of or a mixture of gemcitabine hydrochloride, paclitaxel or its combinations. In one embodiment the polymeric yarns have diameters ranging from 600 to 700 μm, and the nanofibers have diameters ranging from 400-800 nm. And in one embodiment the polymeric yarns have nanofibers loaded with gemcitabine hydrochloride at dosages of between 5 and 10 mg per cm. of length of the SEMS.

In one embodiment of the invention the polymeric yarns are intertwined with stent struts at specific points throughout a full length of the SEMS, the yarns knotted at the distal and proximal ends of the stent struts. Also, in one embodiment the SEMS is adapted to provide a time-released drug elution profile of gemcitabine hydrochloride up to six months. Also, in one embodiment the SEMS is adapted to be deployed endoscopically in a porcine bile duct. Also, in one embodiment the SEMS is adapted to be implanted in a porcine bile duct via an endoscopic procedure and adapted to retain stent patency for a duration of 5 months.

In an alternative aspect of the invention a method for delivering chemotherapeutic drugs is provided, comprising lacing a self-expanding metallic stent (SEMS) with drug-encapsulated polymeric yarns, and implanting the SEMS in an anatomical location where chemotherapeutic drugs are desirable. In one embodiment the method comprises intertwining the polymeric yarns with stent struts at specific locations along a length of the stent. Also, in one embodiment the method comprises forming the polymeric yarns from biodegradable polymers including one or a combination of polycaprolactone, polydioxanone, and polyurethane. Also, in one embodiment the method comprises forming the polymeric yarns from nanofibers loaded with chemotherapeutic drugs, including one of or a mixture of gemcitabine hydrochloride, paclitaxel or its combinations. In one embodiment the method comprises forming the nanofibers with diameters ranging from 400-800 nm. and forming the polymeric yarns from the nanofibers with diameters ranging from 600 to 700 μm. In one embodiment the method comprises loading the nanofibers with gemcitabine hydrochloride at dosages of between 5 and 10 mg per cm. of length of the SEMS.

In one embodiment the method comprises intertwining the polymeric yarns with stent struts at specific points throughout a full length of the SEMS, and knotting the yarns at the distal and proximal ends of the stent struts. Also, in one embodiment the method comprises adapting the loaded nanofibers to provide a time-released drug elution profile of gemcitabine hydrochloride up to six months. Also, in one embodiment the method comprises adapting the SMS to be deployed endoscopically in a porcine bile duct. And in one embodiment the method comprises adapting the SEMS to be implanted in a porcine bile duct via an endoscopic procedure and adapting the SEMS to retain stent patency for a duration of 5 months.

In another aspect of the invention polymeric yarn comprising nanofibers is provided, the yarns loaded with therapeutic material. In one embodiment the therapeutic material is one of or a mixture of gemcitabine hydrochloride, paclitaxel or combinations. In one embodiment the therapeutic material is gemcitabine hydrochloride. And in one embodiment the nanofibers have diameters ranging from 400-800 nm. And the polymeric yarns have diameters ranging from 600 to 700 μm.

### BRIEF DESCRIPTION OF THE SEVERAL VIEWS OF THE DRAWINGS

FIG. 1A is a SEM image of pre-processed nanoyarn.

FIG. 1B is a SEM image of a post-processed nanoyarn.

FIG. 2A shows invitro normal drug release of gemcitabine.

FIG. 2B shows invitro accelerated drug release of gemcitabine hydrochloride from nanoyarns.

FIG. 3A is an optical image of a Type 1 nanostent.

FIG. 3B is an optical image of the nanostent of FIG. 3A crimped.

FIG. 3C is an optical image of the nanostent of FIG. 3A expanded in a delivery device.

FIG. 4A is an optical image of a Type 2 nanostent.

FIG. 4B is an optical image of the Type 2 nanostent crimped.

FIG. 4C is an optical image of the Type 2 nanostent expanded in a delivery device.

FIG. 5 is a schematic diagram of a Type 1 pattern for affixing nanoyarns on stents.

FIG. 6 shows functionality testing of a nanostent in comparison to control SEMS.

FIG. 7 shows remnants of EtO after sterilization.

FIG. 8A is an e Endoscopic image of SEMS in a bile duct of a porcine model.

FIG. 8B is a fluoroscopic image of SEMS in the bile duct of a porcine model.

FIG. 9A shows optical images of an explanted bile duct with a control stent after 3 and 5 months.

FIG. 9B shows optical images of a nanostent after 3 and 5 months.

FIG. 10A shows microscopic images of embedded sections of an explanted bile duct with a control Stent after 3 and 5 months.

FIG. 10B shows microscopic images of embedded sections of an explanted bile duct with a nanostent after 3 and 5 months.

FIG. 11 shows inflammatory analysis of the explanted bile ducts.

FIG. 12A shows representative H&E images of bile duct tissues surrounding the control stent.

FIG. 12B shows representative H&E images of bile duct tissues surrounding the nanostent at 3 and 5 months showing epithelial thickening.

FIG. 13A shows albumin in blood drawn from a nanostent implanted animal at six different time points.

FIG. 13B shows ALP in blood drawn from a nanostent implanted animal at six different time points.

FIG. 13C shows ALT in blood drawn from a nanostent implanted animal at six different time points.

FIG. 13D shows AST in blood drawn from a nanostent implanted animal at six different time points.

FIG. 13E shows CRB in blood drawn from a nanostent implanted animal at six different time points.

FIG. 13F shows total protein in blood drawn from a nanostent implanted animal at six different time points.

FIG. 13G shows total bilirubin in blood drawn from a nanostent implanted animal at six different time points.

FIG. 14A shows representative H&E images of liver tissue from animals implanted with nanostent at 3 and 5 months.

FIG. 14B shows images duodenum tissue from animals implanted with nanostent at 4 and 5 months.

### DETAILED DESCRIPTION OF THE INVENTION

The current gold standard for malignant biliary obstruction in clinics is use of uncovered bare metal SEMS that improve the patency of obstructed bile ducts. However, biliary sludge deposition and eventual tumor in growth within these SEMS compromises long-term patency.

Varieties of stent types that focus mainly on design/shape modification and drug elution have been investigated to partially overcome current drawbacks of biliary stents. Recent work in this area has demonstrated improved benefits for drug-incorporated polymer-coated SEMS over normal SEMS regarding its patency and rate of restenosis. Patent U.S. Pat. No. 9,301,926 B2 describes the invention of a drug-eluting device having a film of thickness from 2 to 1000 μm made of a biodegradable polymer encapsulating chemotherapeutic drugs by solvent evaporation, showing a drug release profile up to 90 days. U.S. Pat. No. 8,999,945 B2 also describes fabrication of a drug-eluting matrix having drug-loaded polymeric fibers via electrospinning, showing a drug release profile of 3 months. However, all these prior arts have established effectiveness of localized drug delivery, though the drug release profiles were only for a shorter duration of up to 3 months. The use of such drug eluting stents in the common bile duct can affect stent patency owing to occlusion at later time periods. Hence to address such limitations, it is important to provide localized drug delivery to the target site and prolong the drug release for sustained durations.

Current research by the inventors addresses most of these limitations through unique inventions relating to use of biodegradable polymeric nanoyarns. Novelty of use of nanoyarns in the instant application is as follows. (i) Each nanoyarn consists of thousands of nanofibers within which the drugs are encapsulated. The very high surface area and the geometrical constraint of the yarn extends drug release times considerably to up to 6 months. (ii) The nanoyarns are considerably stronger than the individual nanofibers and can easily accommodate stent crimping and deployment, without breakage. (iii) The uncovered SEMS integrated with nanoyarns along its length provide long-term patency without biliary sludge deposition.

A nanoyarn integrated SEMS (nanostent) was developed by intertwining Gemcitabine-laden nanoyarns with the stent struts at specific points along the entire length and affixed to the proximal and distal ends of the SEMS. The stent is crimped and loaded inside a delivery device for endoscopic deployment in the bile duct. This nanostent fabricated for biliary stenting satisfies the vital attributes of slow, sustained and localized release of Gemcitabine in the bile duct without causing systemic or organ toxicity; feasibility of crimping and expansion inside the delivery device; functionality performance [stent integrity, fixation effectiveness, and foreshortening]; biocompatibility and sterilizability. The nanostent proved its safety and feasibility when tested in a porcine model, which is demonstrated in this disclosure.

### Embodiments

An important embodiment of the present invention is development of an integrated strategy of utilizing SEMS coupled with the chemotherapeutic drug Gemcitabine eluting nanofibrous yarns that sustain localized drug release at low doses for prolonged durations, to provide dual benefits and establish stent patency for a considerably longer period.

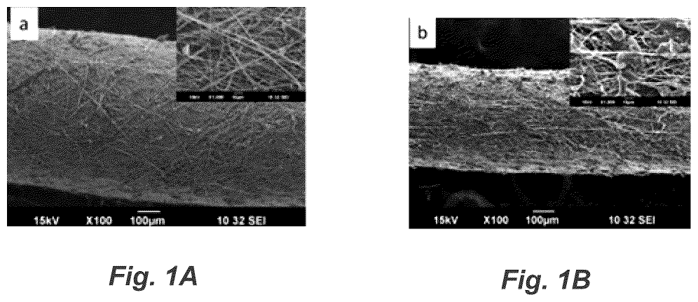

One embodiment in the development of nanostent involves making of continuous nanoyarns laden with chemotherapeutic drugs. In a specific exemplary embodiment, Gemcitabine hydrochloride (GEM) was selected as the chemotherapeutic drug and the biodegradable polymer polycaprolactone (PCL) was selected as the matrix. For this, different drug:polymer ratios were selected (typically 1:1, 1:2, 1:3) for electrospinning onto a translating non-conducting stage set at a low speed. The deposited electrospun nanofibers were twisted and collected as continuous nanoyarns. FIG. 1A represents the SEM image of GEM-loaded PCL yarns (1:2 ratio of drug:polymer) of diameter in the range of 600-700 μm, constituted of fibers of diameter 400-800 nm.

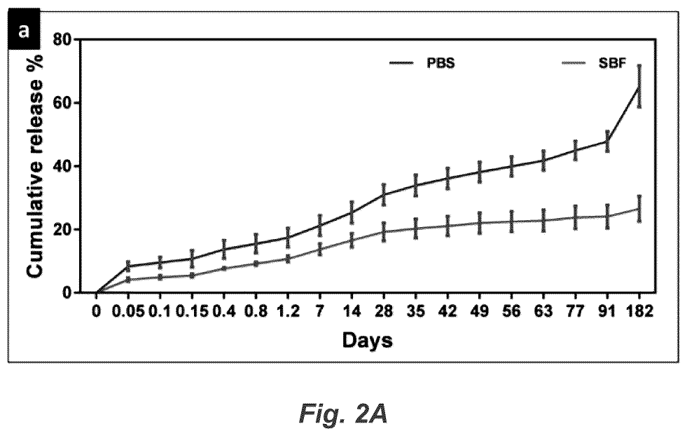

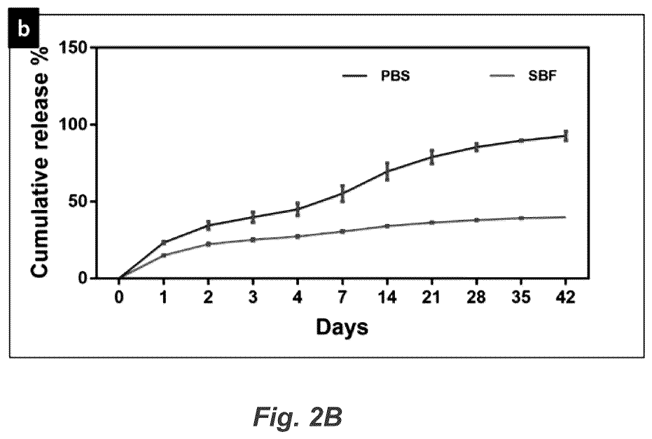

In yet another embodiment, to control drug release, these yarns were post-processed by heating to obtain yarns of diameter in the range of 400-500 μm as shown in FIG. 1B. This enabled tuning the drug release from the yarns, which was then tested in vitro in phosphate-buffered saline and simulated bile fluid (SBF) under normal [37° C., 200 rpm, pH=7.4] and accelerated [50° C., 80 rpm, pH=7.4] conditions. The drug release was measured by HPLC, at a retention time of 3.8 min and a wavelength of 275 nm. FIGS. 2A and 2B) depict normal and accelerated drug release profiles monitored in PBS and SBF. The results prove that under both normal and accelerated conditions, release kinetics reveal the slow and sustained release of Gemcitabine from PCL. Accelerated release data clearly show that drug release can be prolonged for more than six months.

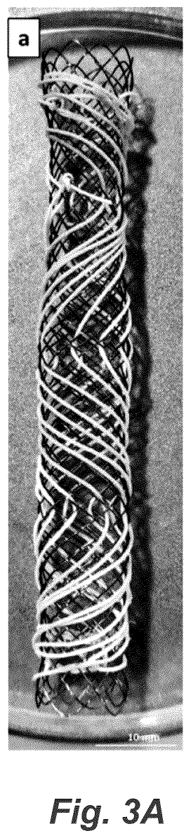

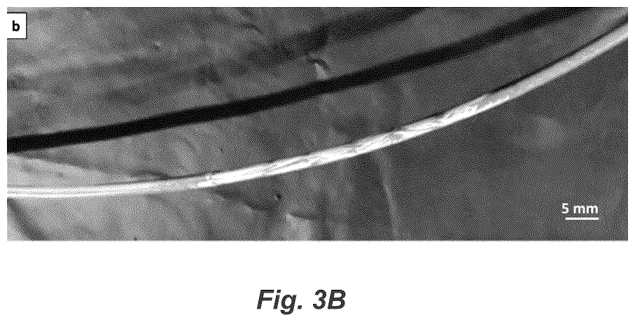

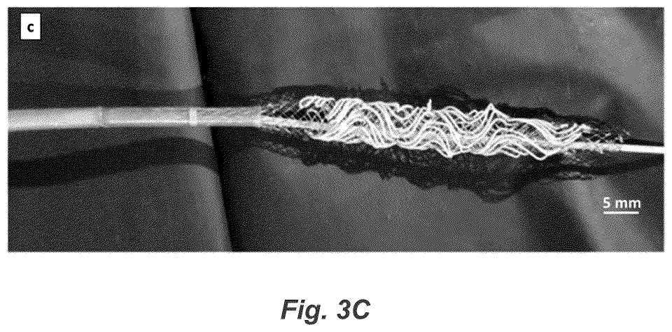

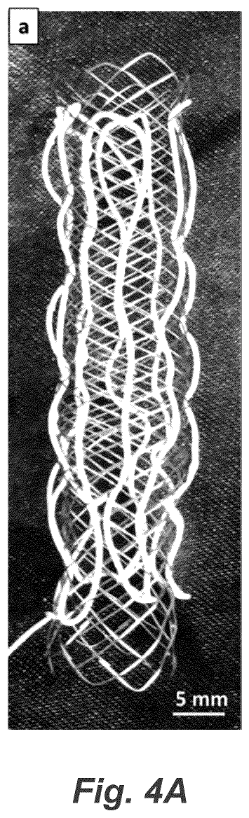

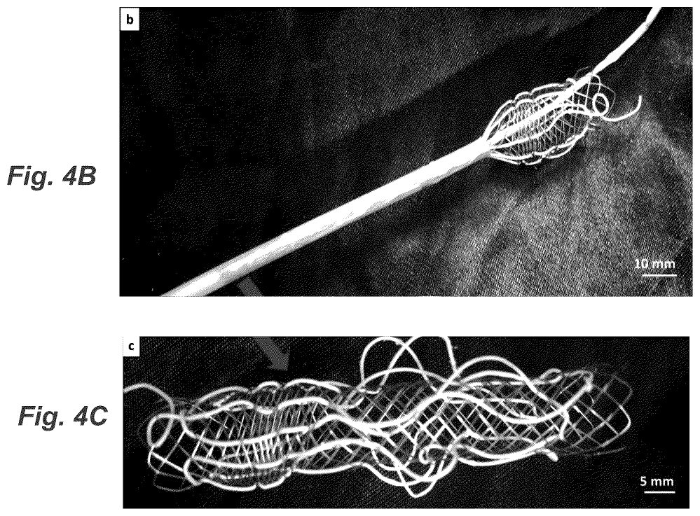

In this particular embodiment, nanostents were developed by intertwining gemcitabine-laden nanoyarns with the stent struts at specific points along the entire length of the stent. FIG. 3A and FIG. 4A depict the images of two types of nanostents developed by affixing the nanoyarns on to the stents in two different patterns. FIG. 3B is an optical image of the nanostent of FIG. 3A crimped. FIG. 3C is an optical image of the nanostent of FIG. 3A expanded in a delivery device.

Specifically, in Type 1 geometry (FIG. 3A), the nanoyarn was overlayed with the stent in a sinusoidal pattern and was intertwined with the struts at the crest and trough regions of the sinusoidal geometry. This nanoyarn was then affixed to the stent at its proximal and distal ends, with a clearance of typically 5 mm for a 6 cm long stent. This sinusoidal pattern can be defined by the amplitude being equivalent to half the stent diameter and frequency varying with the stent length. Typically, for a stent of length 5 cm and diameter 1 cm, the amplitude will be 0.5 cm and frequency will be one sine wave. In Type 2 geometry (FIG. 4A), for the same length of the nanoyarn as used in Type 1 geometry, the nanoyarn was overlayed with the stent in a sinusoidal pattern, such that the amplitude of the sine wave is typically zero and the frequency is one sine wave for a stent of length 5 cm and diameter 1 cm. The nanoyarn was intertwined with the struts at the node of the wavy pattern. This nanoyarn was also affixed to the stent at its proximal and distal ends, with a clearance of typically 5 mm for a 6 cm long stent. The above two types of integration allowed a maximum drug dose of ˜10 mg per cm of the SEMS.

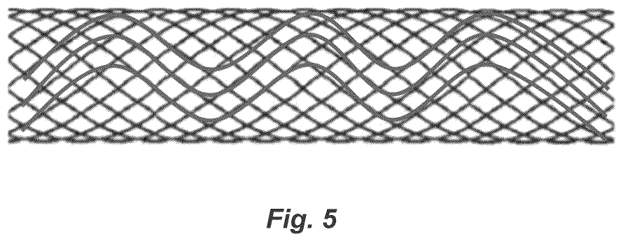

Both crimping and expansion of these stents were performed after its incorporation within a 10 Fr delivery device, as depicted in FIG. 4B and 4C respectively. it was found that the ability to crimp Type 1 nanostent was easier than Type 2 nanostent owing to the aligned pattern of affixing the yarns on the stent. Later, the deployment potential of these stents was evaluated by pushing it out from the delivery device. While Type 2 nanostent was deployed with deformity, Type 1 stent deployed without any structural damage as shown in FIGS. 3C and 4C, pointing to the inefficiency of Type 2 nanostent for further use. FIG. 5 shows the schematic representation of Type 1 pattern which was utilized for fabricating the nanostent.

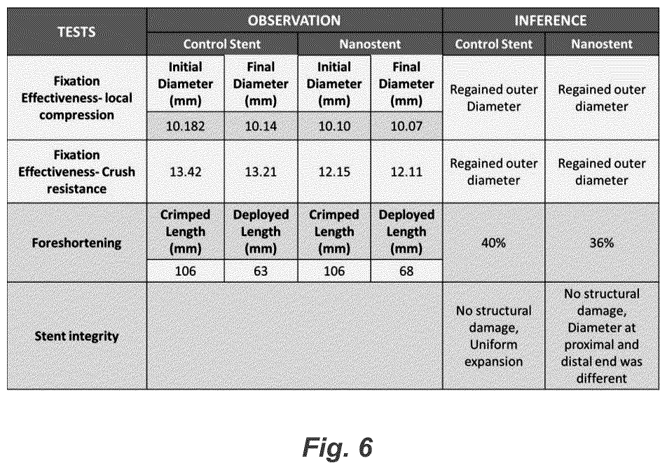

The functionality assessment of the nanostent was performed as per ISO standards (ISO 25539) by evaluating the fixation effectiveness by local compression and crush resistance, foreshortening, and stent integrity and compared with the control stent (Commercially available SEMS). FIG. 6 shows that the results of all the tests for nanostent and Control stents were comparable.

Fundamental design parameters of the stent such as the number of cells per unit length, the lumen diameter and length of the stent are also evaluated for the nanostent. The number of cells per unit length of the stent remains the same (50 cells per unit length of the stent) for both the control and nanostent, as the latter is only a surface modified version of the former one. The lumen diameter of the control stent is measured to be 10 mm and its length 60 mm, and these values are not altered significantly by integrating the nanoyarn with the SEMS in fabricating the nanostent. Thus, the lumen diameter of the nanostent is measured to be nearly 10 mm, while the stent length is in the range of 60-65 mm. Likewise, the pore size of the nanostent is comparable to that of the control stent as the modification is done via overlaying the nanoyarns above the stent strut and intertwined at specific points. Thus, the coverage of the SEMS is not hampered by integrating it with the nanoyarn, thereby defining the nanostent as a drug-eluting uncovered self-expanding metallic stent.

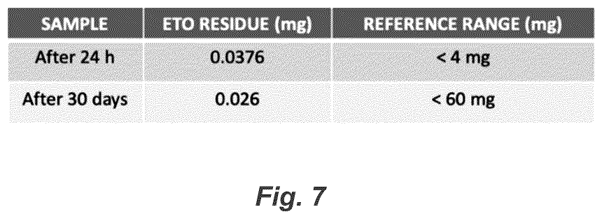

ETO sterilization is a well-acclaimed method that is used for sterilizing medical devices and equipment. In this embodiment, ETO sterilization was adopted and parameters were optimized such that the residual ETO content is within admissible limits as per ISO standards. Accordingly, ETO sterilization of the stent prototype was done for 2 h, which was followed by degassing for 14 h. Any remnant ETO was then analyzed by GCMS [Method: USEPA 5021 A] after 24 h and 30 days of incubation at 37° C. based on ISO 10993-7. FIG. 7 shows the concentration of remnant ETO after the two specified durations, viz., 24 h and 30 d. Remnant ETO levels were found to be within permissible limits.

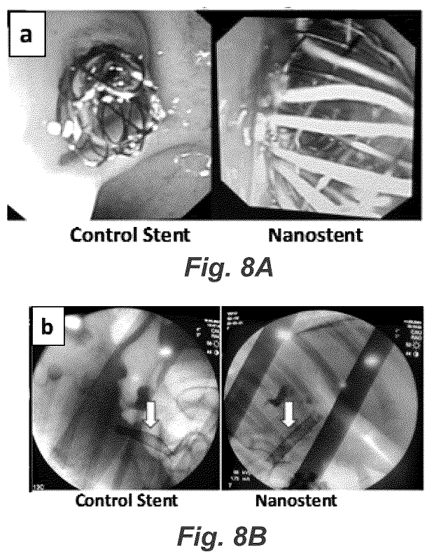

To assess the feasibility and safety of the developed device, in vivo implantation of nanostent was done in the porcine bile duct under fluoroscopic guidance via ERCP procedure. In this embodiment, in a total of 14 animals (Yorkshire-desi crossbred pigs), nanostents and control SEMS (SEMS without nanoyarns) were implanted and analyzed for two-time points (3 and 5 months). To confirm stent placement in the bile duct of the animal, endoscopic and fluoroscopic images were taken as shown in FIGS. 8A and 8B respectively. The fluoroscopic images of the control and nanostent implanted bile duct reveal the proper deployment of the stents in the bile duct. This points to the feasibility of implantation of the nanoyarn-integrated SEMS in vivo.

Endoscopic follow-up was performed in the animals at regular intervals. Blood and bile were collected for analysis of the drug content at intermittent time periods. These were then tested for the presence of GEM and its metabolite by HPLC. GEM and its metabolite DFDU could not be detected in blood up to the limit of its detection i.e., 300 ng. This implies that the drug does not enter systemic circulation. The drug content in bile was also undetectable, owing to the drug release happening in a continuously flowing bile fluid. However, the presence of remnant drug could be measured in the nanoyarns taken from explanted stents at 3 and 5 months, to be ˜1000 μg and 50 μg respectively. This confirms that the nanoyarn integrated with the stent can provide a localized drug release over prolonged time periods.

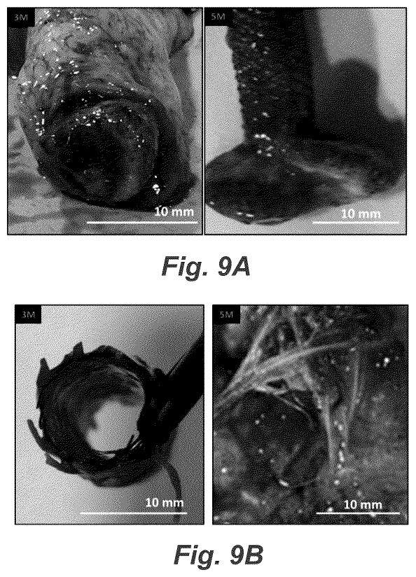

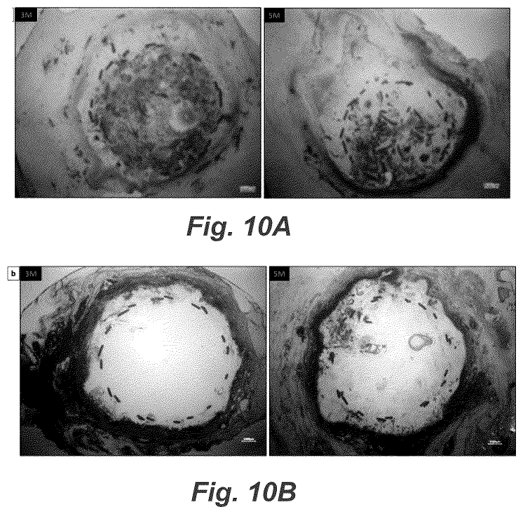

After completion of the implantation period, viz., 3 and 5 months, the animals were euthanized, and the bile duct was explanted. Pictograms of the explanted bile duct with the Control and nanostents for the two-time points investigated are shown in FIGS. 9A and 9B. The lumen of the nanostent was patent and not occluded, like in the control stent. This was further confirmed using H&E staining of the sections of the explanted bile duct. FIGS. 10A and 10B represent the microscopic images of the H&E-stained sections, which confirm the open lumen of the nanostent at both time points, implying stent patency, in contrast to the occluded Control stent. The term ‘stent patency’ refers to the lumen of the stent which remains open without occlusion, from the time of stent insertion till the completion of end time points or euthanasia of the animal.

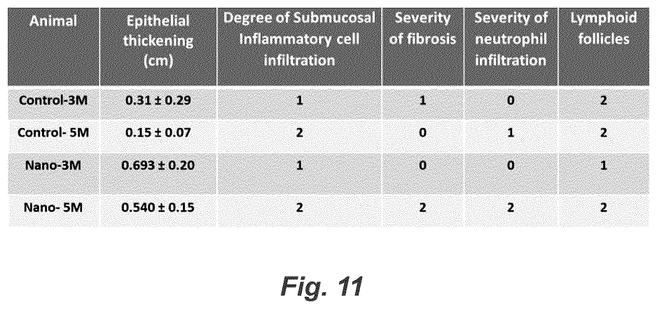

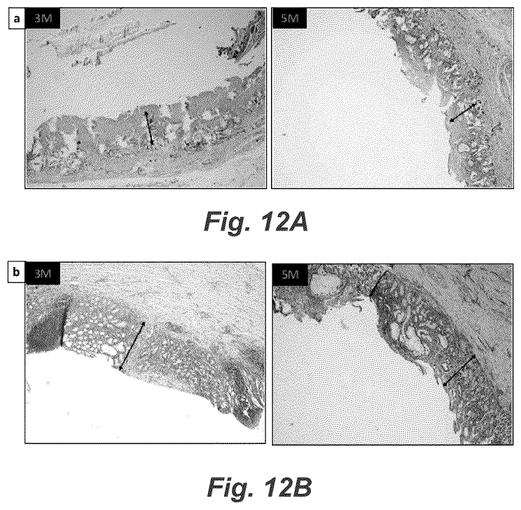

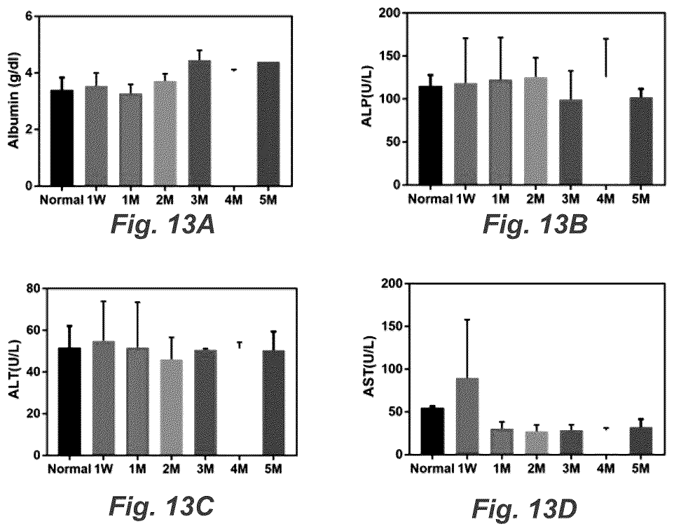

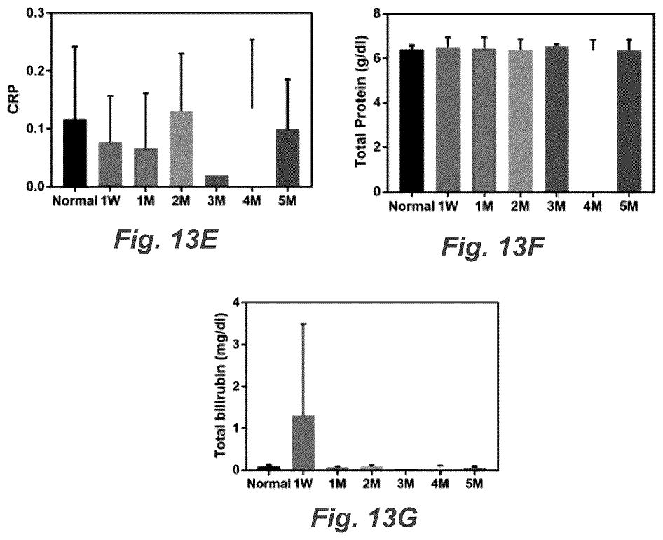

FIG. 11 shows inflammatory analysis of the explanted bile duct based on neutrophil and inflammatory cell infiltration, epithelial thickening, severity in fibrosis, and the presence of lymphoid follicles. The nanostent did not induce significant inflammation at 3 months, similar to the control stent, whereas a mild inflammatory response was observed in the 5-month group. The H&E images of the tissue surrounding the nanostents are shown in FIGS. 12A and 12B which shows the absence of severe epithelial thickening, as in control stent. FIGS. 13A through 13G show biochemical analysis for liver function tests, wherein it was found that Albumin, ALT, ALP, CRP, and total protein were all within the normal range for all the time points tested. However, the values of AST and bilirubin were high in the first week, which became normal with time.

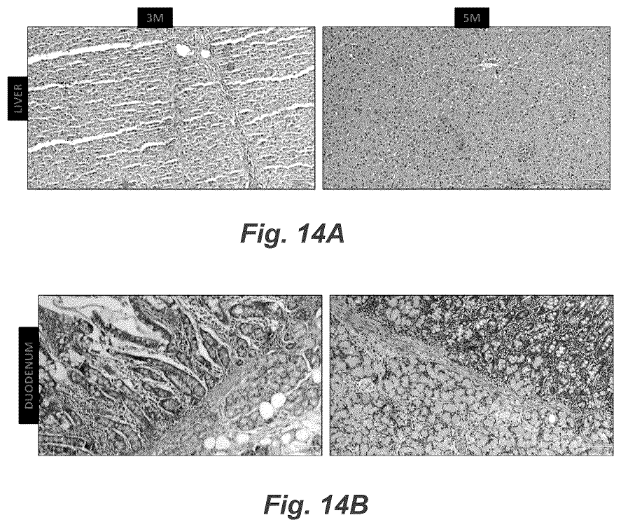

Histological analysis of the vital tissues, viz., liver and duodenum, at two time points of implantation of nanostents, viz., 3 and 5 months, showed the absence of inflammatory cell infiltration, mucosal hyperplasia or necrosis as shown in FIGS. 14A and 14B. This implies that the nanostent is biocompatible. All these results prove the safety and feasibility of the nanostent for biliary stenting applications.

Technical advantages of the present invention over other uncovered metallic SEMS are highlighted and summarized as follows:
The stent retains its capacity to expand in the presence of the drug loaded nanoyarn;—Proven by FIGS. 3A through 3C in comparison with FIGS. 4A through 4C, and 6 (ISO standards ISO 25539).
nanoyarn wound in the abluminal side of the stent releases the drug slowly at least for a period of 6 months, without obstructing the lumen of the stent, thus ensuring long term patency. (FIGS. 8A, 8B, 9A and 9B show long term patency). Drug content in the bile and blood could not be detected. However, the concentration of remnant drug measured in the nanoyarns taken from explanted stents at 3 and 5 months was found to be ˜1000 μg and 50 μg respectively, confirming that the nanoyarn integrated stent could provide a localized drug release over prolonged time periods.
nanoyarns integrated on the stent did not obstruct the flow of bile into the common bile duct; (FIGS. 10A and 10B)
The stent assembly ensures localized drug delivery into the biliary lumen without toxic consequences. (FIGS. 13A through 13G and FIGS. 14A and 14B).

A skilled person will understand that the embodiments illustrated and described in this application are all exemplary, and not limiting. There may be many other embodiments within the scope of the invention. The scope is limited only by the claims.

1. A self-expanding metallic stent (SEMS) with drug-encapsulated polymeric yarns intertwined with stent struts at specific locations along a length of the stent.

2. The SEMS of claim 1 wherein the polymeric yarns comprise biodegradable polymers including one or a combination of polycaprolactone, polydioxanone, and polyurethane.

3. The SEMS of claim 1 wherein the polymeric yarns have nanofibers loaded with chemotherapeutic drugs, including one of or a mixture of gemcitabine hydrochloride, paclitaxel or its combinations.

4. The SEMS of claim 3 wherein the polymeric yarns have diameters ranging from 600 to 700 μm, and the nanofibers have diameters ranging from 400-800 nm.

5. The SEMS of claim 1 wherein the polymeric yarns have nanofibers loaded with gemcitabine hydrochloride at dosages of between 5 and 10 mg per cm. of length of the SEMS.

6. The SEMS of claim 3 wherein the polymeric yarns are intertwined with stent struts at specific points throughout a full length of the SEMS, the yarns knotted at the distal and proximal ends of the stent struts.

7. The SEMS of claim 5 adapted to provide a time-released drug elution profile of gemcitabine hydrochloride up to six months.

8. The SEMS of claim 1 adapted to be deployed endoscopically in a porcine bile duct.

9. The SEMS of claim 1 adapted to be implanted in a porcine bile duct via an endoscopic procedure and adapted to retain stent patency for a duration of 5 months.

10. A method for delivering chemotherapeutic drugs comprising:
lacing a self-expanding metallic stent (SEMS) with drug-encapsulated polymeric yarns; and
implanting the SEMS in an anatomical location where chemotherapeutic drugs are desirable.

11. The method of claim 10 comprising intertwining the polymeric yarns with stent struts at specific locations along a length of the stent.

12. The method of claim 10 comprising forming the polymeric yarns from biodegradable polymers including one or a combination of polycaprolactone, polydioxanone, and polyurethane.

13. The method of claim 10 comprising forming the polymeric yarns from nanofibers loaded with chemotherapeutic drugs, including one of or a mixture of gemcitabine hydrochloride, paclitaxel or its combinations.

14. The method of claim 13 comprising forming the nanofibers with diameters ranging from 400-800 nm. and forming the polymeric tarns from the nanofibers with diameters ranging from 600 to 700 μm.

15. The method of claim 1 comprising loading the nanofibers with gemcitabine hydrochloride at dosages of between 5 and 10 mg per cm. of length of the SEMS.

16. The method of claim 13 comprising intertwining the polymeric yarns with stent struts at specific points throughout a full length of the SEMS, and knotting the yarns at the distal and proximal ends of the stent struts.

17. The method of claim 15 comprising adapting the loaded nanofibers to provide a time-released drug elution profile of gemcitabine hydrochloride up to six months.

18. The method of claim 10 comprising adapting the SMS to be deployed endoscopically in a porcine bile duct.

19. The SEMS of claim 10 comprising adapting the SEMS to be implanted in a porcine bile duct via an endoscopic procedure and adapting the SEMS to retain stent patency for a duration of 5 months.

20. Polymeric yarn comprising nanofibers, the yarn loaded with therapeutic material.

21. The polymeric yarns of claim 20 wherein the therapeutic material is one of or a mixture of gemcitabine hydrochloride, paclitaxel or combinations.

22. The polymeric yarn of claim 20 wherein the therapeutic material is gemcitabine hydrochloride.

23. The polymeric yarn of claim 20 wherein the nanofibers have diameters ranging from 400-800 nm. And the polymeric yarns have diameters ranging from 600 to 700 μm.
